(19) United States
(12) Patent Application Publication
(10) Pub. No.: US 2019/0184089 A1
(43) Pub. Date: Jun. 20, 2019

(54) INSULATED AUTO-INJECTOR CONTAINER

(71) Applicants: ULRIKE H M ZIEGNER, PALOS VERDES ESTATES, CA (US); DAVID LAITURI, WAYLAND, MA (US); JON EDWIN HOKANSON, PALOS VERDES ESTATES, CA (US)

(72) Inventors: ULRIKE H M ZIEGNER, PALOS VERDES ESTATES, CA (US); DAVID LAITURI, WAYLAND, MA (US); JON EDWIN HOKANSON, PALOS VERDES ESTATES, CA (US)

(21) Appl. No.: 16/192,988

(22) Filed: Nov. 16, 2018

Related U.S. Application Data
(60) Provisional application No. 62/587,396, filed on Nov. 16, 2017.

(51) Int. Cl. A61M 5/00

(52) U.S. Cl. CPC A61M 5/002; A61M 2005/2006

(57) ABSTRACT

An insulated, soft, durable fabric pouch sized and adapted to hold one or more epinephrine auto-injectors, or insulin injectors, the pouch including preferably a gel type insulation surrounding at least a major part of the injector(s), a rapid-opening top flap to facilitate immediate and easy availability of the injector for grasping and using by the user.

### FIELD OF INVENTION

The invention relates generally to cases for medication delivery devices such as auto-injectors used for injection of epinephrine, insulin and other drugs that are temperature sensitive.

### BACKGROUND

Cases, pouches or other types of containers for medication delivery devices such as auto-injectors used for injection of epinephrine and insulin are well-known. However, no known conventional epinephrine or insulin auto-injector case or container provides a soft fabric housing made of a durable material, such as nylon, with at least one insulated chamber that protects the epinephrine or insulin from degradation by exposure to temperatures that are outside the useful temperature range of the drug that is contained in the auto-injector container.

### SUMMARY

Embodiments of an auto-injector container or pouch according to the present invention overcome the drawbacks of known auto-injector containers and pouches by providing a soft fabric housing or pouch that includes one or more insulated chambers for one or more injectors to facilitate rapid availability of each injector for grasping by the user, and a pop-open flap to provide secure containment of each injector within an insulated, protective housing or case.

Most preferably the present invention is in the form of a pouch having two chambers, one chamber for each auto-injector. Preferably the flap is of a design that provides for rapid access to the auto-injector, and is sized and configured to house two injectors, although it may also be adapted for a single injector or for more than two injectors. In another preferred embodiment a temperature measuring device or strip of material is included on the interior of the pouch to monitor and record the temperature inside of the pouch so that the user can be informed if the auto-injector has been heated to a degree that renders its epinephrine, insulin or other contained liquid or gel (the contained material) to have reduced effectiveness or to be ineffective. During use for containing epinephrine the pouch should be kept at room temperature, and not pre-cooled or pre-refrigerated. When used for containing insulin, (or other liquid that may safely be pre-cooled or pre-refrigerated), the disclosed pouch can be pre-cooled by placing it in a refrigerator, ice chest, and or by any other means to lower the temperature of the pouch and its contained insulation material to a temperature below ambient temperature. When the pouch is pre-cooled, without insulin or other contained liquid or gel that may safely be pre-cooled or pre-refrigerated, inside the pouch, and the pouch is later used with the contained insulin or other contained appropriate liquid or gel, then the effective time or period during which the contained material remains within a prescribed, safe temperature range is extended beyond the effective time that would result from use when the starting temperature is at or above ambient temperature.

Several embodiments, features, aspects, and advantages of the invention will become better understood with regard to the following description, appended claims and accompanying drawings.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a front perspective view of a preferred embodiment, insulated container or pouch for a conventional EpiPen® brand epinephrine auto-injector or various brands of insulin injectors;

FIG. 2 is an open flap front view, a side view and a top view of the FIG. 1 insulated container or pouch;

FIG. 3 is a closed flap front view, side view and side cross-sectional view of the FIG. 1 insulated container or pouch;

FIG. 4 is a front view of an alternate embodiment insulated pouch;

FIG. 5 is an illustration of illustrative instructions for use of the FIG. 4 embodiment; and,

FIG. 6 illustrates four different versions of insulin auto-injector that may be used with the present insulated container or pouch.

Reference symbols or names that may be used in the Figures to indicate certain components, aspects or features shown therein. Reference symbols common to more than one Figure indicate like components, aspects or features shown therein.

### DETAILED DESCRIPTION

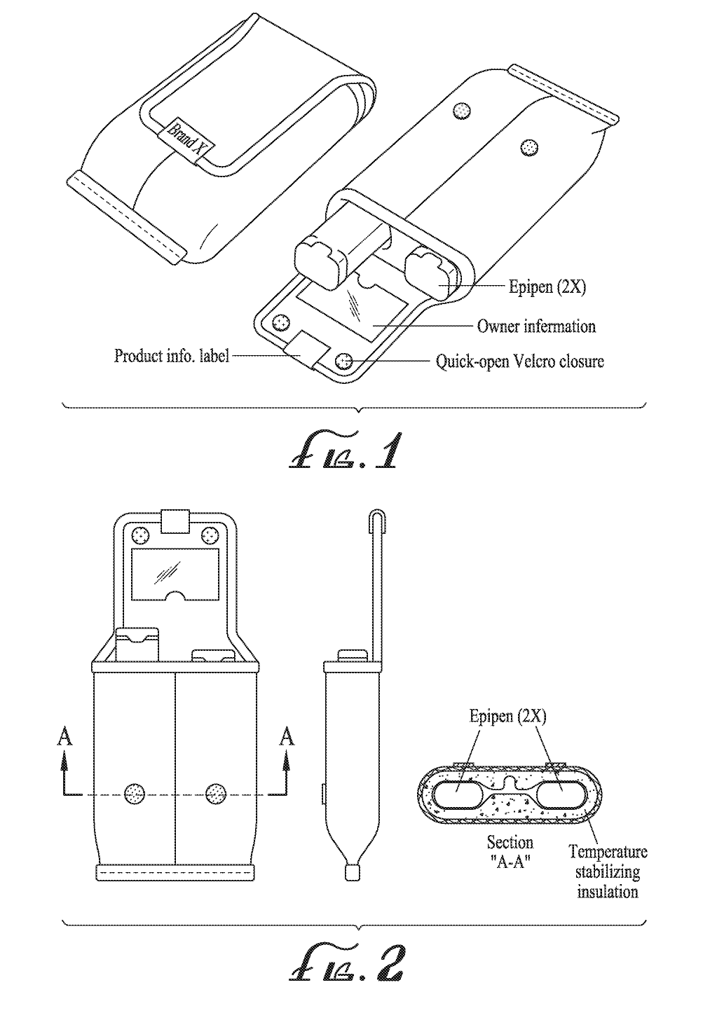

With reference to FIGS. 1-3 a first preferred embodiment housing or case or pouch for auto-injectors is described. The case is adapted to hold two EpiPen® brand epinephrine auto-injectors or two insulin auto-injectors in a side-by-side relationship.

FIG. 1 shows structural features in a perspective view of a preferred embodiment, soft fabric, and insulated pouch adapted to hold two injectors, in a top flap closed and top flap open configuration.

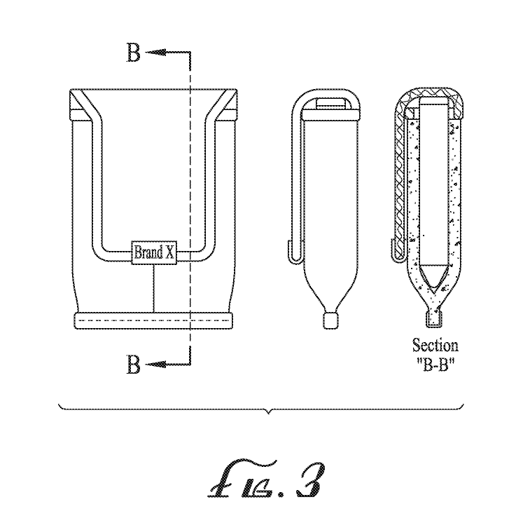

Three FIG. 2 show structural features in an open flap front view, side view and top view of the FIG. 1 insulated case. The pouch may be of various sizes, configured to hold one or more epinephrine auto-injectors or various brands of insulin injectors or other items that are temperature sensitive. The preferred pouch is generally rectangular in shape, has a rectangular body, a close bottom end, and open top end and a flap. The top flap that overlies the top, open end of the pouch body as show. The pouch is made of a flexible, strong, durable material, such as nylon. The bottom of the pouch is closed, preferably by sewing. The FIG. 2 embodiment is sized and configures to hold two epinephrine auto-injectors. The pouch includes pockets for holding the injectors, and includes one or more insulation-filled cavities between the outer surface of the body of the pouch and the inner surface of the cavities into which the auto-injectors are positioned.

The insulation material may also be referred to as temperature stabilizing insulation. The insulation material may be any of numerous, conventional insulation materials, so long as the pouch functions for its intended purpose of providing the minimum temperature stabilization as described below, that is, maintaining a conventional EpiPen® brand epinephrine auto-injector a 120° F., hot car example, at least about three hours of protection against reaching an unacceptable internal temperature in a surrounding environment of about 120° F., such as could be experienced in a hot car parked in daylight in a warm climate, for example. The most preferred insulation material is a gel made of cellulose gum (cellulose ether and cellulose polymer) and water, of the type included in a sold in Koolit CMC Brand Gel Packs, available from Cold Chain Technologies, Inc., 29 Everett St., Holliston, Mass. 01746.

As shown n FIG. 2 the insulation surrounds the injectors on the four sides of the pouch body. The pouch may also be configured to have insulation-filled pockets in the top flap and at the bottom of the pouch to provide additional temperature stabilization. As shown in FIG. 2 the top flap is in an open position and the temperature stabilizing insulation is shown inside the pouch, surrounding the auto-injector.

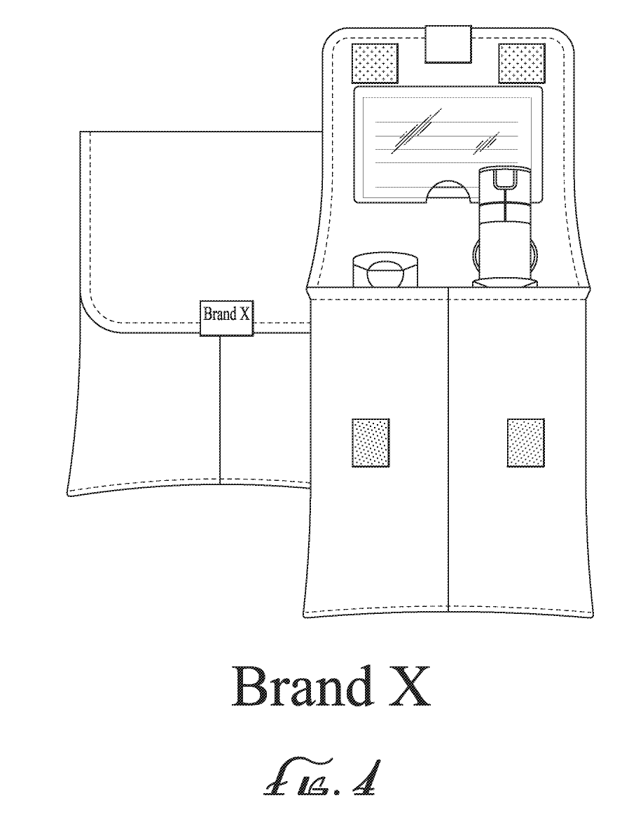

FIG. 3 shows structural features of the pouch and enclosed auto-injector with a closed flap front view, side view and side cross-sectional view of the FIG. 1 insulated case embodiment.

As shown and described with respect to FIGS. 1-3, the disclosed insulated pouch is adapted to encase, preferably, two EpiPen® brand injectors. The pouch stabilizes the epinephrine's temperature throughout the day, thereby extending the useful life of the injector and minimizing the possibility of degrading the encased epinephrine when the case is exposed to temperature extremes. Exemplary tests conducted on a preferred embodiment pouch, for an ambient temperature of 120° F., such as could be found in a hot car in the southern part of the United States, more than 3 hours of protection against the epinephrine injector reaching an unacceptable internal temperature as compared to about 3 minutes for the same injector that is exposed to this temperature and without being in the insulated pouch.

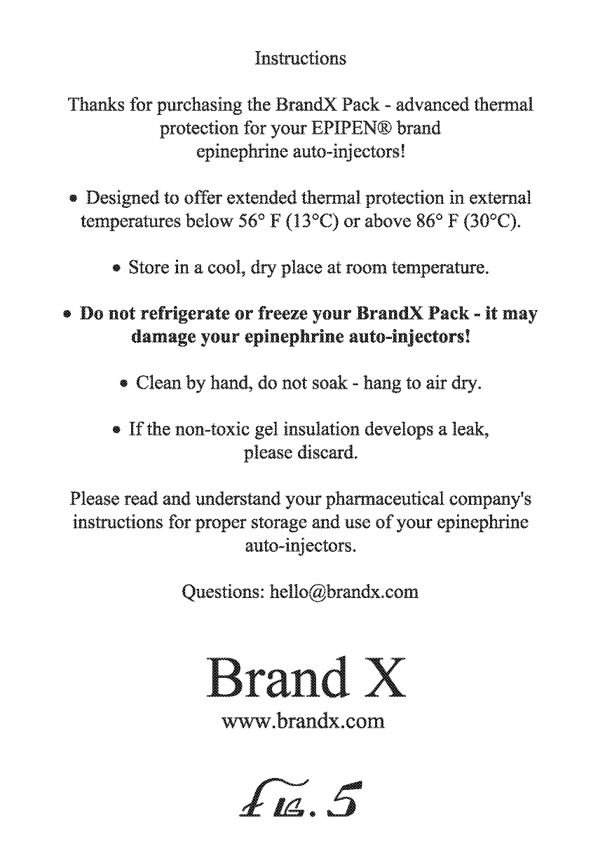

Referring to FIG. 4, alternate embodiments of the insulated pouch are shown. The FIG. 4 embodiments illustrate pouches of different colors: bright safety orange and a shade of blue that is believed would not stand out in many typical situations of use, for a user who does may not want to draw attention to the pouch. The FIG. 4 pouches are shown with one in a front view and the other in a back or rear view. The FIG. 4 pouches also show a information/identification tag positioned on the inside of the top flap, and two sets of Velcro brand hook and loop fasteners positioned on the outside of the pouch body and correspondingly placed fasteners places on the inside surface of the top flap. These fasteners function to retain the injectors/pens, while also providing for rapid opening of the pouch for immediate access to the injectors.

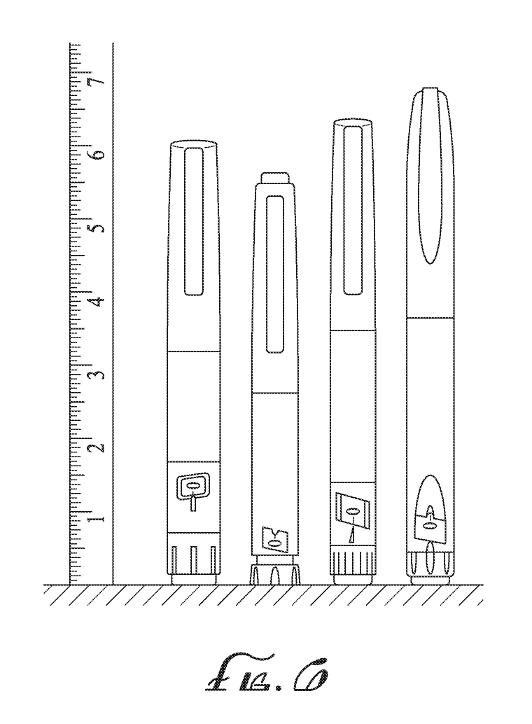

FIG. 5 illustrates typical instructions and warnings for use.

FIG. 6 shows four conventional insulin pens, along side a ruler. As is readily apparent, the largest of the insulin pens is approximately 6 inches high. Each of these pens may also be used with the FIGS. 1-4 insulated pouch. The presently disclosed auto-injector pouch may also be used with other brands of insulin auto-injectors or insulin “pens”, as commonly they are commonly referred to. While various brands of insulin auto-injectors are known, as exemplified in FIG. 6, the presently described pouch that is preferably sized for use with EpiPen® brand injectors can be used for only those insulin auto-injectors that will fit into the cavity that has predetermined dimensions sufficiently large to hold the insulin auto-injector. The presently described pouch may also be made in different sizes and with different numbers of cavities so that it can hold various sizes of and various numbers of insulin auto-injectors. For example, the principles of the present invention and the general shape and constituents of the presently described pouch can be modified to hold a single insulin pen.

While the disclosed pouch should not be pre-cooled or pre-refrigerated when used to contain EpiPen® brand injectors, when used for containing insulin auto-injectors (insulin pens), pre-cooling or pre-refrigerating the pouch will function to extend the period during when the insulin will remain within safe temperature limits.

Also, conventional, low-conductivity, high heat capacity insulation positioned inside of the pouch, as shown, for example, in FIGS. 2 and 3, protects the encased epinephrine, insulin or other material in both hot and cold extreme ambient environments. The pouch is preferably made of a rugged, durable nylon material that is sewn, sized and adapted for active, mobile use. The insulated pouch can be “recharged” by leaving it in a normal, room temperature environment, as well as by placing it in a low temperature environment, such as a refrigerator or cooler to bring its temperature back to a normal room temperature, as would be understood by those skilled in this field. The present insulated pouch, in a sewn form can be produced in a cost-effective manner both in low and in high volume quantities. The insulated case can also have advertising, branding (such as shown in FIG. 4), instructional material, safety notices, emergency phone numbers or other information placed on or included in or on the fabric exterior, such as on the inner side of the flap, as shown in FIG. 4. The present insulated pouch can also be produced in a wide range of colors, examples of which are described and shown with regard to FIG. 4.

Although specific embodiments of the invention have been described, various modifications, alterations, alternative constructions, and equivalents are also encompassed within the scope of the invention.

The specification and drawings are, accordingly, to be regarded in an illustrative rather than a restrictive sense. It will, however, be evident that additions, subtractions, deletions, and other modifications and changes may be made thereunto without departing from the broader spirit and scope of the invention as set forth in the claims.

What is claimed is:

1. An epinephrine auto-injector pouch comprising:
a durable, nylon soft fabric housing having a length, width, height, top end flap, closed bottom end;
two cavities extending along the length of the housing;
insulation material positioned around said cavities, inside of said housing and adapted to maintain the interior of said case at ambient temperature for a predetermined period; and,
each of said cavities sized and configured to encase an epinephrine auto-injector.
